(19) United States
(12) Patent Application Publication
(10) Pub. No.: US 2023/0139070 A1
(43) Pub. Date: May 4, 2023

(54) DETECTION OF WATER CONTENT IN TISSUE

(71) Applicant: United Kingdom Research and Innovation, Swindon Wiltshire (GB)

(72) Inventors: Pavel Matousek, Oxfordshire (GB); Nicholas Stone, Exeter (GB); Adrian Ghita, Exeter (GB)

(73) Assignee: United Kingdom Research and Innovation, Swindon Wiltshire (GB)

(21) Appl. No.: 17/912,328

(22) PCT Filed: Feb. 17, 2021
(86) PCT No.: PCT/GB2021/050387

§ 371 Date: Sep. 16, 2022

(30) Foreign Application Priority Data
Mar. 19, 2020 (GB) 2004010.1

(51) Int. Cl. A61B 5/00

(52) U.S. Cl. CPC A61B 5/4875; A61B 5/0075

(57) ABSTRACT

There are disclosed methods and apparatus for measuring water content in tissue in-vivo, for example in sub-surface or sub-cutaneous tissue of a human or animal subject. The measurement may be made through diffusely scattering overlying tissue such as skin tissue, by: directing probe light to an entry region on a surface of the overlying tissue; collecting said probe light from a collection region on the surface of the overlying tissue, the collection region being spatially offset from the entry region, the collected probe light comprising probe light inelastically scattered into the Raman OH stretching bands by water present in the sub-surface tissue; detecting, in the collected probe light, one or more first spectral features of the probe light inelastically scattered into the Raman OH stretching bands; and measuring water content in the sub-surface tissue using the one or more first spectral features.

The present invention relates to methods and apparatus for measuring water content in tissue, for example in vivo tissue of a human or animal subject. Such tissue may be subcutaneous, and the methods and apparatus may enable such measurements to be made non-invasively. Other chemical signatures may be measured at the same time, for example to better characterise aspects of the tissue such as the presence of cancerous lesions.

### INTRODUCTION

X-ray techniques are frequently used to detect sub-surface cancerous tissues in patients, for example using X-ray mammography. However, such X-ray techniques use potentially harmful ionizing radiation, and provide little or no specificity to chemical properties or compositional information of the tissue being studied.

Magnetic resonance imaging can provide more compositional information of sub-surface tissues, but the equipment is complex and expensive both to obtain and operate.

It would be desirable to address problems and limitations of the related prior art.

### SUMMARY OF THE INVENTION

Embodiments of the invention use Raman spectroscopy techniques which probe sub-surface tissues of a subject or patient, such as spatially offset geometries (SORS) and transmission geometries to detect the presence of elevated water content in such tissues.

Such information may then be used for a variety of purposes, including to non-invasively determine the presence of cancerous tissue or lesions in vivo. This detection of elevated water content may in particular make use of high wavenumber areas of the Raman spectrum, and advantageously may be combined with information from other areas of the Raman spectrum, especially at lower wavenumbers in the so-called “fingerprint” region, from which can be determined other or related chemical properties of the tissue. For example, the detection of cancerous tissues may be carried out using a combination of water detection in the high wavenumber Raman region, and a measure of one or more other indicative chemical species in the low wavenumber, or fingerprint, Raman region.

The Raman signature of water is typically very weak in the lower wavenumber areas of the Raman spectrum usually used for spatially offset (SORS) and transmission geometries in Raman spectrometry, but strong Raman water bands appear in the high wavenumber region, for example the Raman OH stretching band located around a wavenumber shift of 3400 cm⁻¹. With suitable optimization of probe light wavelength and detection arrangements a strong water signature can be detected simultaneously with other high wavenumber bands originating from non-water components giving a non-water signature, such as the strong Raman C—H stretching bands found around a wavenumber shift of 3100 cm⁻¹.

A comparison or combination of the water and non-water signatures then provides an improved measure of water content, for example representing a ratio of water to soft tissue content in the measured tissue, thereby providing a rapid and safe screening technique for water content abnormalities, or for more particular tissue characteristics or types such as sub-surface cancerous tissues. A particular example of such tissue which can be detected in this way is cancerous tissue within for example in breast soft tissues.

According to some aspects, the invention provides a method of detecting or measuring, in vivo, and optionally non-invasively, water content in a sub-surface tissue of a human or animal subject, for example elevated water content indicative of cancerous tissue. The sub-surface tissue may for example be tissue which is beneath at least the epidermis, and optionally also beneath the dermis layers of skin, for example being subcutaneous tissue which is beneath the skin of the subject.

The water content of the sub-surface tissue can be detected or measured through diffusely scattering overlying tissue. For example the diffusely scattering overlying tissue may comprise, for example, at least the stratum corneum, and optionally the whole epidermis, the whole epidermis and dermis, or the whole of the skin tissue. The diffusely scattering overlying tissue may also include tissue beneath the skin which overlies the sub-surface tissue to be measured.

The method may then comprise: directing probe light to an entry region on a surface of the overlying tissue; collecting said probe light from a collection region on the surface of the overlying tissue, the collection region being spatially offset from the entry region, the collected probe light comprising probe light inelastically scattered into water-sensitive portions of the high wavenumber Raman spectrum indicative of the presence of water in the sub-surface tissue; detecting, in the collected probe light, one or more first spectral features of the probe light inelastically scattered into the high wavenumber Raman spectral features indicative of the presence of water; and measuring or detecting water content in the sub-surface tissue using the one or more first spectral features.

In particular, the high wavenumber Raman spectral features indicative of the presence of water may be the Raman OH stretching bands. These bands may extend at least from a wavenumber shift of about 2800 cm⁻¹ to a wavenumber shift of about 3700 cm⁻¹ (for example within 50 cm⁻¹ of 100 cm⁻¹ of these boundaries), and result from interaction of the probe light with free of bound water generally in the liquid phase. The Raman OH stretching bands may typically comprise a first spectral peak in intensity or power at about 3400 cm⁻¹ (for example with the apex of the peak lying between about 3300 cm⁻¹ and 3500 cm⁻¹), although the exact position of the observed peak may depend on factors such as sample temperature, sample chemical environment, response characteristics of the spectrometer or other measurement device, and optical transmission properties of components within the apparatus used.

The one or more first spectral features may for example comprise one or more of: an area under at least a portion of the first spectral peak; and a magnitude of at least a portion of the first spectral peak.

The collected probe light may further comprise probe light inelastically scattered into reference portions of the Raman high wavenumber region which are characteristic of the sub-surface tissue, but which are not or which are only minimally sensitive to the amount of water present in the tissue. Suitable such baseline portions may be found for example in the Raman CH stretching bands which arise from C—H bonds present in the sub-surface tissue.

The method may then comprise detecting, in the collected probe light, one or more second spectral features, of probe light inelastically scattered into these reference high wavenumber regions which are not, or are only minimally, sensitive to the presence of water, such as the Raman spectral CH stretching bands. The method may then comprise measuring the water content of the sub-surface tissue using both the one or more first spectral features, and the one or more second spectral features, in combination. For example, the water content of the sub-surface tissue maybe measured using the one or more first spectral features normalised using the one or more second spectral features.

The Raman CH stretching bands may extend at least from a wavenumber shift of about 2800 cm⁻¹ to a wavenumber shift of about 3100 cm⁻¹ (for example within 50 cm⁻¹ of these boundaries). The Raman CH stretching bands may conveniently provide a second spectral peak at about 2900 cm⁻¹ (for example within 100 cm⁻¹ of this point). The one or more second spectral features may comprise one or more of: an area under at least a portion of the second spectral peak; and a magnitude of at least a portion of the second spectral peak, and/or various other measures.

As noted above, determination of properties of the sub-surface tissue may make use of detection of high wavenumber Raman spectral features in combination with detected Raman spectral features at lower wavenumbers, and in particular in the fingerprint region. Typically, the Raman fingerprint region may be taken to extend up to a wavenumber shift of about 1800 cm⁻¹.

To this end, the probe light may further comprise probe light inelastically scattered into the Raman fingerprint region by one or more chemical components of the sub-surface tissue, the method then also comprising detecting, in the collected probe light, one or more third spectral features of probe light inelastically scattered into the Raman fingerprint region. The third spectral features may then be used to measure or detect chemical components of the sub-surface tissue using the one or more third spectral features, and properties of the sub-surface tissue such as a detection of cancerous tissue may be determined by combining detected or measured water content with one or more chemical properties detected using the third spectral features.

In implementing the techniques described, the entry and collection regions are disposed on opposite sides of the sub-surface tissue, so that the collected light contains Raman spectral features representative of a bulk of the tissue between the entry and collection regions, including the sub-surface tissue. Although the sub-surface tissue may be disposed directly between the entry and collection regions, other configurations of the entry and collection regions may be used, for example with these regions disposed approximately at right angles with respect to a central region of the sub-surface tissue, or in other ways.

In other implementations, the techniques may involve separately detecting said one or more spectral features in the collected probe light for each of a plurality of different spatial offsets between said entry and collection regions, in a SORS configuration. Measuring water content of the sub-surface tissue from the spectral features may then comprise associating the spectral features from each of said plurality of different spatial offsets with a different depth or distribution of depth beneath the surface. The detected spectral features may then be used by combining said spectral features from said different spatial offsets to determine a separate measure of water content for each of one or more depths or distributions of depth beneath the surface.

The entry and collection regions may be configured in different ways, for example with the or each collection region comprising one or more segments which are disposed around a centrally disposed collection region, for example with each collection region being in the form of an annulus, or in an inverse-SORS arrangement in which the or each entry region comprises one or more segments which are located around a centrally disposed collection region, with the or each entry region being in the form of an annulus.

In order to provide suitable probing and depth selection of the sub-surface tissue, the entry and collection regions may be spatially offset by a plurality of offsets lying in the range from 1 mm to 50 mm, and more preferably in the range from 3 mm to 20 mm.

Whether a SORS technique with one or more offsets, or a transmission Raman technique are used, the sub-surface tissue to be detected may lie beneath the surface of the subject (typically this surface may be defined by the surface of the skin or the stratum corneum of the subject) by least twice the diffuse scattering transport length of probe light in the sub-surface tissue, for example at least 1 mm or at least 2 mm or at least 5 mm beneath the surface of the subject.

Having measured or determined water content of the sub-surface tissue, an indication of whether the sub-surface tissue is cancerous may be generated from the measured water content, and optionally also from one or more of the third spectral features detected in the collected probe light of the Raman fingerprint region or chemical properties derived therefrom. For example, the one or more third spectral features detected in the collected probe light of the Raman fingerprint region may be indicative of the presence of characteristic changes within nucleic or amino acids, lesion related calcifications, protein to lipid ratios, or changes in protein confirmations associated with dysfunctional tissues.

The described measuring of water content of the sub-surface tissue may result in an output which can take a wide range of values from a high level to a low level (or zero level) of water content, or the output may be a binary indication of elevated water content of the sub-surface tissue, for example with respect to a predetermined threshold.

The method may also comprise generating a two dimensional map of one or more of determined parameters, such as measured water content in the sub-surface tissue, and an indication of the sub-surface tissue being cancerous, and so forth. The two dimensional map then corresponds to the surface of the overlying tissue and indicates the relevant property of the sub-surface tissue beneath. For example, such a map could be generated from repeated measurements of water content or repeated indications of the sub-surface tissue being cancerous, taken at different positions across the surface.

Although the described techniques are particularly useful for detecting or measuring water content and other properties of the sub-surface tissue, it should be understood that some Raman signature of the overlying tissue will typically also be mixed in with the signal from the sub-surface tissue, and cannot generally be excluded altogether. However, use of both the SORS and transmission Raman techniques described below allow for a significant degree of depth selection to take place, thereby reducing the contribution from the overlying tissues. Furthermore, in many situations the variations in water content of the tissue being studied arise largely or strongly from variations of the sub-surface tissues, as in the case of probing for sub-surface cancerous tissue in breasts and other body regions. In such situations detected variations in water content may largely be attributed to variations in the sub-surface tissue.

Aspects of the invention also provide apparatus suitable for or arranged to implement the described methods. For example, the invention provides apparatus for measuring, in vivo, and optionally non-invasively, water content in a sub-surface tissue or a subcutaneous tissue of a human or animal subject, through diffusely scattering overlying tissue, comprising: a light source for generating probe light; delivery optics arranged to direct probe light to an entry region on a surface of the overlying tissue; collection optics arranged to collecting said probe light from a collection region on the surface of the overlying tissue, the collection region being spatially offset from the entry region; a spectrometer arranged to detect, in the collected probe light, one or more first spectral features of a portion of the probe light inelastically scattered into Raman OH stretching bands by water present in the sub-surface tissue; and an analyser arranged to determine water content in the sub-surface tissue from the one or more first spectral features, for example to detect an elevated water content.

The spectrometer may be arranged to detect, in the collected probe light, one or more second spectral features of a portion of the probe light inelastically scattered into Raman CH stretching bands by C—H bonds present in the sub-surface tissue; and the analyser may then be arranged to determine water content in the sub-surface tissue from the one or more first spectral features and the one or more second spectral features.

The spectrometer may be arranged to detect, in the collected probe light, one or more third spectral features of a portion of the probe light inelastically scattered into the Raman fingerprint region by chemical components present in the sub-surface tissue; and arranged to detect the chemical components in the sub-surface tissue from the one or more third spectral features.

The analyser may then be arranged to determine properties of the sub-surface tissue, such as whether the tissue is cancerous, from both the determined water content and the detected chemical components.

In some arrangements, the delivery optics and the collection optics may be arranged such that, in use, they lie on opposite sides of the sub-surface tissue, or spaced widely apart on the surface so as to permit a Raman scattering from a bulk of the tissue proximal to the entry and collection regions, or broadly disposed between the regions, to be probed, in a transmission or forward scattering configuration.

In other arrangements, the apparatus further comprises an offset driver arranged to provide a plurality of different offset spacings between the entry and collection regions, preferably such that the light collected by the collection regions has been broadly backscattered to the collection regions. The apparatus may then be arranged to separately detecting said one or more spectral features in the collected probe light for each of a plurality of different spatial offsets between said entry and collection regions. The analyser may then be arranged to measure water content in the sub-surface tissue from the spectral features by associating the spectral features from each of said plurality of different spatial offsets with a different depth or distribution of depth beneath the surface.

Aspects of the apparatus such as analysis of detected spectral features, consequent determination of properties of the sub-surface tissue, and generation of indicators or alarms responsive to such determined properties, either by the analyser or other elements, may conveniently be implemented using computer program code implemented on one or more suitable computer systems. Such computer systems may typically comprise one or more microprocessors to execute the program code, and volatile and/or non-volatile memory to store the program code, input data such as spectral data received from the spectrometer, and output data such as particular detected spectral features, indications and measures of water content, and indications and measures of other properties such as an indication of elevated water content in the sub-surface tissue.

To this end, the invention also provides one or more computer readable media comprising computer program code arranged to carry out such aspects of the invention.

### BRIEF DESCRIPTION OF THE DRAWINGS

Embodiments of the invention will now be described, by way of example only, with reference to the accompanying drawings, of which:

FIG. 1 shows, schematically, apparatus for measuring, in vivo, water content of a sub-surface or subcutaneous tissue of a human or animal subject, with the inset graph depicting characteristics of a Raman spectrum obtained by the spectrometer 26;

FIG. 2 shows in more detail aspects of a high wavenumber region of the inset graph of FIG. 1;

FIGS. 3a to 3c show examples of offset geometries between entry and collection regions on a surface of the subject of FIG. 1;

FIG. 4 shows some different ways in which entry and collection regions may be disposed around tissue of the subject of FIG. 1;

FIG. 5 depicts an experimental setup for demonstrating efficacy of the described techniques;

FIG. 6 shows high wavenumber regions of Raman spectra obtained during drying of a tissue sample using the experimental setup of FIG. 5;

FIG. 7 demonstrates preparation of a sample for use in the experimental setup of FIG. 5;

FIGS. 8a and 8b show maps of water content obtained using the experimental setup of FIG. 5 to scan across the surface of the sample of FIG. 7, with FIG. 8c showing a difference map between these; and

FIG. 9 illustrates a method according to the invention.

### DETAILED DESCRIPTION OF EMBODIMENTS

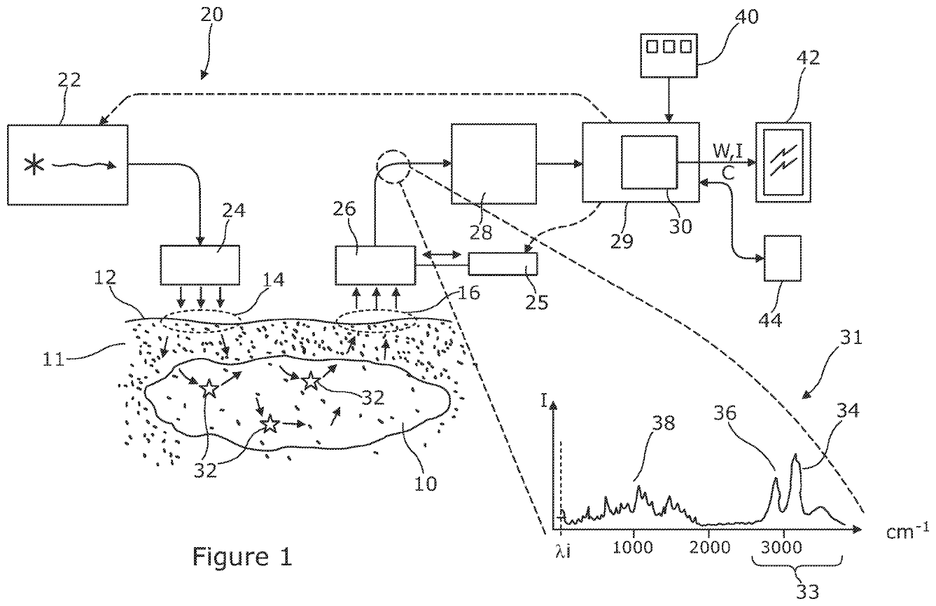

Referring now to FIG. 1 there is shown schematically apparatus 20 for measuring water content of a region of sub-surface tissue 10 of a human or animal subject, through a diffusely scattering overlying tissue 11 providing an exposed surface 12 of the subject. For implementations where the surface 12 through which the water content is measured by the apparatus is the surface of skin or the stratum corneum of the subject, or of some other external surface such as the surface of the cornea, the measurement of water content may then be described as non-invasive. If the diffusely scattering overlying tissue is, or includes the skin of the subject, and the sub-surface tissue is beneath this skin then the measurement may be described as a sub-cutaneous measurement of water content. However, in other implementations the surface 12 may be a surface of surgically exposed tissue, or a surface accessed using a non-surgical procedure such as endoscopy.

The apparatus comprises a light source 22 arranged to form a beam of probe light, and delivery optics 24 arranged to direct the beam of probe light to an entry region 14 on the surface 12 of the subject. Collection optics 26 are arranged to collect probe light, which has been scattered within the overlying tissue 11 and the sub-surface tissue 10, from a collection region 16 which is spatially offset from the entry region 14. Optional relative movement of the entry and collection regions, to provide a plurality of different such spatial offsets, may be provided by an offset driver mechanism 25, which may form part of or be combined with or be arranged to control the delivery optics and/or the collection optics.

A spectrometer 28 then receives the collected light, and detects spectral features in the collected light which arise from inelastic, and in particular Raman, scattering of the probe light within the sub-surface tissue 10. Data relating to the detected spectral features are then used by an analyser 30 to measure and optionally output a measure of water content W of the sub-surface tissue 10, or of one or more volumes or regions of the sub-surface tissue 10, from the detected spectral features, as discussed further below. The analyser 30 may also be arranged to generate and output measures C of other chemical components of the sub-surface tissue using the detected spectral features, and/or other indicators of tissue type I based on one or more of water content, other chemical components and so forth, such as an indicator that the currently probed sub-surface tissue may be cancerous.

The analyser 30 may be comprised within a controller 29 used for other aspects of control and operation of the apparatus, for example executing in software on a microprocessor of such a controller 29, or could be located or implemented elsewhere for example distant from the optical and control aspects of the apparatus.

Using the offset driver, the apparatus may be arranged to separately detect Raman spectral features for each of a plurality of different offsets between the entry and collection regions, so as to provide a more depth selective measurement of water content, and/or water content measurements at multiple depths or multiple profiles of depth which may be overlapping.

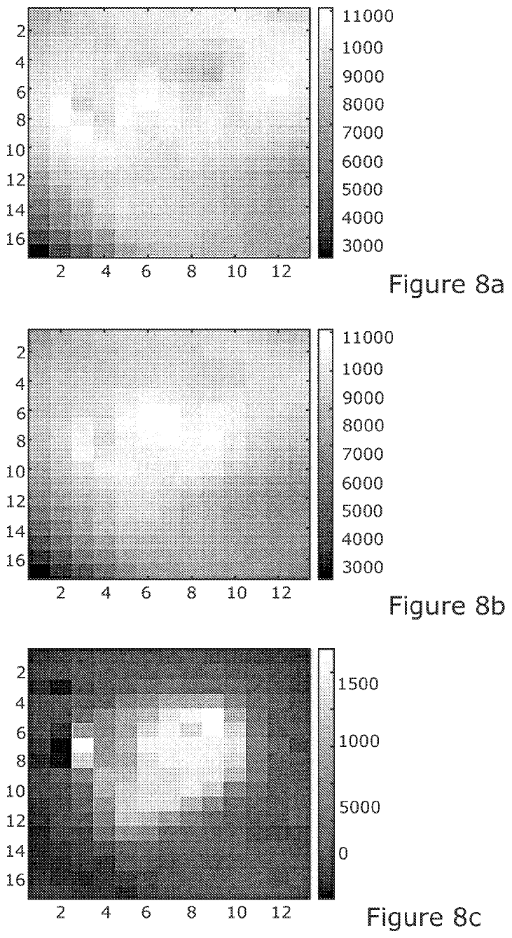

Although not depicted in FIG. 1, the apparatus may also be arranged to laterally translate the analysed tissue relative to the delivery and collection optics. In this way, by making measurements of water content Wand optionally other output measures such as C and I, at multiple positions across the surface 12, a map of such measurements can be generated, such as the maps illustrated in FIGS. 8a-8c. From such a map, particular sub-surface tissue regions which are of interest, for example due to their elevated water content, can more easily be localised and identified by a user of the equipment. Particular uses of such techniques are to permit a user to more accurately identify the position and extent of cancerous tissue or lesions, or to determine cancer margins or the presence or margins of oedematous tissues.

The light source 22 may typically be a near infrared or visible red laser, emitting a beam of probe light in the near infrared or visible red region of the electromagnetic spectrum. For example, a laser emitting at a wavelength between 630 nm and 720 nm, or more particularly around 680 nm may be used. Using a light source in this visible wavelength region may be advantageous in enabling conventional Raman spectral arrangements using CCD detectors or similar to detect both fingerprint and higher wavenumber regions of the Raman spectrum as discussed below.

The delivery optics 24 may be provided by one or more suitable optical fibres and/or lenses arranged to form the beam of probe light into a suitably configured entry region 14 on the surface of the sample. The collection optics 26 may also be provided by one or more suitable optical fibres and/or lenses to define the collection region 16 on the surface of the sample and to collected probe light from this region and deliver it to the spectrometer 28. In some arrangements, the entry and collection regions may conveniently be provided by a combined hand-held probe, for example using suitably spaced and arranged optical fibres to deliver to and collect from the entry and collection regions.

The spectrometer 28 may be provided in various ways such as using a dispersive spectrometer, or by suitable optical filters in combination with photodetectors, or in other ways, in order to detect particular spectral features in the collected light.

Aspects of spectral features detected by the spectrometer 28 are illustrated in FIG. 1 at graph 31 which has a vertical axis of relative intensity or power, and a horizontal axis of Raman wavenumber shift. In this graph, the wavelength of the probe light generated by the light source 22 is depicted as λᵢ. Typically, this wavelength of light may be suppressed or substantially removed by the collection optics 26, before the collected light is received by the spectrometer 28, for example using a suitable optical filter, so that there is no corresponding central peak shown in the graph 31.

In order to measure water content of the sub-surface tissue 10 from the detected spectral features, the detected spectral features include first spectral features of portions of the collected probe light which have been inelastically scattered into a high wavenumber region 33 of the Raman spectrum, typically above about 2700 or 2800 cm⁻¹, and in particular into water-sensitive portions of this region, for example scattered into the Raman OH stretching bands 34 by liquid water present in the sub-surface tissue 10. Raman scattering events within the sub-surface tissue 10 are depicted in FIG. 1 by reference numeral 32. The measurement of water content may also use detected second spectral features of portions of the collected probe light which have been inelastically scattered into other areas of the high wavenumber spectrum which can act as reference portions of the spectrum through being insensitive to water content, for example the Raman CH stretching bands 36 by C—H bonds present in the sub-surface tissue 10. The second spectral features may arise from a variety of chemical species, but these species may typically may be dominated by proteins and lipids in tissue.

Chemical components of the sub-surface tissue will also inelastically scatter the probe light into the fingerprint region 38 of the Raman spectrum, typically seen below wavenumber shifts of about 1800 cm⁻¹. The detected spectral features may therefore also comprise third spectral features of portions of the collected probe light scattered into the fingerprint region, which can then be used to measure chemical components other than water in the sub-surface tissue, such as the calcifications in breast tissue, or changes characteristic of cancer within Raman signatures of nucleic and amino acids.

The controller 29 may be used to provide control and/or monitoring of various elements and aspects of the apparatus, for example of the light source 22 and the offset driver 25. An input 40 may be used to provide user input or control instructions to the apparatus by connection to the controller, and a display 42 may be used by the apparatus to output operational information. The determined water content W or water content profile(s), and/or data derived from such water content information, and determined other chemical components C and/or date derived from such information, including one or more indicators of tissue type I, may also be presented to a user on the display 42, and/or output to another entity using a data connection 44. Of course, the input 40 and display 42 could be combined into a single touch screen display if desired.

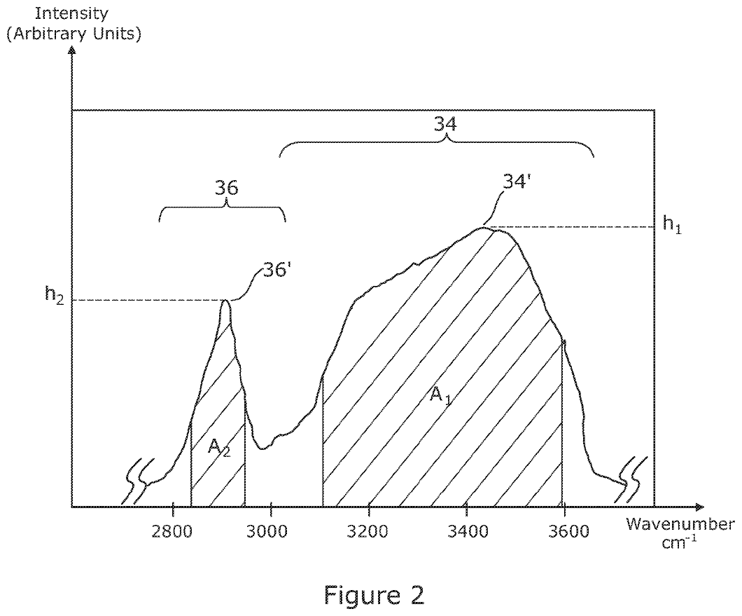

FIG. 2 depicts a high wavenumber region 33 of spectral features detected by spectrometer 28 and processed by analyser 30 in order to measure water content W of the sub-surface tissue. The high wavenumber region depicted extends from a wavenumber shift (relative to the zero shift of the probe light generated by light source 22) of about 2800 cm⁻¹ to about 3700 cm⁻, but the detected high wavenumber region may extend beyond these bounds, especially to the upper end. In FIG. 2 the lower wavenumber, fingerprint region 38 is not shown.

The collected probe light which gives rise to the detected high wavenumber spectral region 33 comprises probe light inelastically scattered into Raman OH stretching bands 34 by water present in the sub-surface tissue. These Raman OH stretching bands typically extend from a wavenumber shift of about 2800 cm⁻¹ to a wavenumber shift of about 3700 cm⁻¹, and are made up of Raman spectral features arising from OH groups engaged in various types of hydrogen bonding. However, these stretching bands more particularly exhibit a first spectral peak 34′ with an apex at about 3400 cm⁻¹ and which can be seen as dominant over other spectral features over the region of about 3000 cm⁻ to about 3600 cm⁻.

Various first spectral features relating to the OH stretching bands may therefore be conveniently measured and used by processor 30 for determining or measuring water content in the sub-surface tissue. Such first spectral features may include for example an area under at least a portion of the first spectral peak 34′, depicted in FIG. 2 as area A₁, a magnitude of at least a portion of the first spectral peak 34′ such as apex intensity h₁, or in various other ways.

The collected probe light which gives rise to the detected high wavenumber spectral region 33 also comprises probe light inelastically scattered into Raman CH stretching bands 36 by C—H bonds present in the sub-surface tissue, for example in proteins, carbohydrates and other organic molecules. These Raman CH stretching bands typically extend from a wavenumber shift of about 2800 cm⁻¹ to a wavenumber shift of about 3100 cm⁻¹, and are made up of Raman spectral features arising from CH groups in various binding situations. However, these stretching bands more particularly exhibit a second spectral peak 36′ with an apex at about 2900 cm⁻¹ and which can be seen as dominant over other spectral features over the region of about 2800 cm⁻¹ to about 3000 cm⁻¹.

Various second spectral features relating to the CH stretching bands may therefore be conveniently measured and used by processor 30 for providing data to support in measuring water content in the sub-surface tissue. Such second spectral features may include for example an area under at least a portion of the second spectral peak 34′, depicted in FIG. 2 as area A₂, a magnitude of at least a portion of the second spectral peak 36′ such as apex intensity h₂, or in various other ways.

Although it may be practical to measure water content of the sub-surface tissue using spectral features only in portions of the high wavenumber spectrum sensitive to water such as the Raman OH stretching bands, measurements of these features may vary widely depending on aspects such as variations in the degree of coupling of probe light into and out of the overlying tissue, and between the overlying and sub-surface tissues, diffusive and elastic scattering properties and refractive index variations within the tissues, stability of the light source 22 and other aspects of the apparatus of FIG. 1, and so forth. To this end, the measurement of water content in the sub-surface tissue may advantageously combine the one or more first spectral features relating directly to the water content, and the one or more second spectral features which are insensitive or minimally sensitive to water content but instead relate to other aspects of the tissue such as CH bonds.

In some embodiments, the analyser 30 may therefore measure water content W of the sub-surface tissue only using the one or more first spectral features arising from the OH stretching bands, for example by a linear relationship between one or more such features and a measure of water content. However, in other embodiments the processor may use both the first and second spectral features in measuring water content. This may be carried out by using the second spectral features to normalise the first spectral features. For example, a simple ratio of magnitudes of first and second spectral features such as W=A₁/A₂ or W=h₁/h₂ could be used. Any such measure of W may of course be converted into suitable units as required, such as ratio weight of water per corresponding total weight of sub-surface tissue, which may require some further processing of the measure of water content for example using a calibration curve or table.

Other ways in which the processor may derive a measure of water content from the spectral features include various chemometric analysis techniques.

Having calculated a measure of water content, this may be used in various ways by the analyser 30 or other aspects of the apparatus. For example, the measure of water content may be used to generate a determination of elevated water content in the sub-surface tissue for example by comparing the measure to a threshold, or determination that the sub-surface tissue is cancerous. Such a determination could be used for example to trigger or generate an audible or visual alarm or indicator, to better enable a user to perceive the determination for example as the subject is scanned using the apparatus.

As mentioned above, the fingerprint region of the Raman spectrum shown as 38 in the inset graph of FIG. 1 may be used to detect the presence or concentration of a wide range of chemical species in the sub-surface tissue. To this end, the analyser 30 may also be arranged to receive, from the spectrometer 28, third spectral features arising in the fingerprint region 38, and to use this third spectral features in generating further indications of properties of the sub-surface tissue. For example, some particular chemical species which may be detected in this way which may be indicative of cancerous tissue include nucleic and amino acids, and lesion related calcifications. Detected levels of one or more such compounds may be output to a user, or combined with the measure water content in order to provide an improved determination or indicator of cancerous tissue.

For example, if the water content determined from the first and second spectral features is denoted W, and the level of a particular chemical species characteristic of a particular cancerous tissue is denoted C, then a binary indication I of potentially cancerous tissue could be set to “yes” if W×C>T, and “no” otherwise, where T is a suitable predetermined threshold.

The proportion of scattering of the probe light within the sub-surface tissue which is inelastic Raman scattering, compared with the proportion of scattering which is elastic scattering is typically very small, usually with a difference of many orders of magnitude, and especially when the sample is highly scattering as is typically the case with human tissue and many other application areas. As a consequence, most photons of probe light are not Raman scattered in the sub-surface tissue 10. However, nearly every photon of probe light which is Raman scattered within the sub-surface tissue is also subsequently scattered elastically a large number of times in that tissue and the overlying tissue 11, giving rise to a random walk of the photon before emerging from the surface to be collected by the collection optics.

The average path of this random walk through the sub-surface tissue and the overlying tissue, between the entry region and the collection region, depends on the spatial offset between these regions. It can be seen that for larger spatial offsets the average depth of the path will be deeper within the tissues.

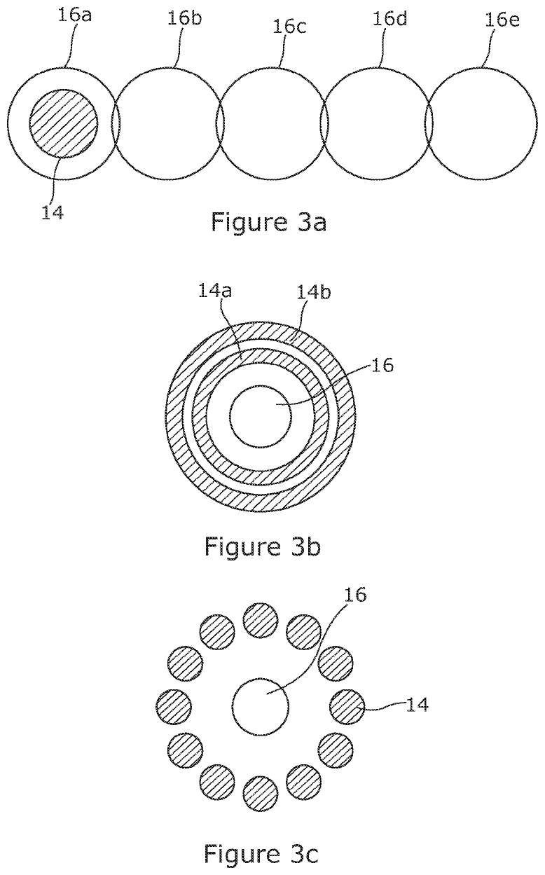

Using this principle, the spacing between the entry and collection regions can be controlled or adjusted by the apparatus 20 in order to control the distribution of depths at which the Raman scattering occurs. This technique is referred to as spatially offset Raman spectroscopy (SORS), and is discussed in detail in WO2006/061565 and WO2006/061566, the contents of which are incorporated herein by reference for all purposes, including for illustrating how characteristics of the sub-surface tissue may be determined at particular depths and profiles of depth within the tissue. According to the present invention, such characteristics may include water content, measured using the high wavenumber region of the Raman spectrum, and other measurements of other chemical components using the low wavenumber or fingerprint region. Some ways in which Raman spectral features or related information from different spatial offsets may be combined to derive characteristics of the sub-surface tissue selected for one or more depths or one or more profiles of depths, are discussed in the above patent publications, but may include simple subtraction schemes for example in which the spectral features for a small or null offset are subtracted from those of one or more larger offsets, or more complex multivariate analysis, such as principle component analysis in which statistical relationships between detected spectral features at multiple offsets are used to derive water content and other chemical characteristics at a depth, profile of depth, or multiple such depths or profiles of depth. According to the principles of spatially offset Raman spectroscopy, therefore, the entry and collection regions may be of various sizes and shapes, and for any particular spatial offset these regions may each be formed by single contiguous or multiple discrete segments on the surface of the sample. Some examples of such regions are depicted in FIGS. 3a to 3c.

In FIG. 3a, an entry region 14 is provided at a fixed position, and multiple collection regions 16a-16e are provided at increasing spatial offsets from the single entry region. Optionally, one of the illustrated collection regions 16a is coincident with, or overlapping with the entry region 14, so as to form a zero offset or null spacing. This zero offset can conveniently provide a signal representative of the surface of the sample, for compensating signals derived from larger offsets. This can be done, for example, by subtracting Raman features detected for the null spacing from Raman features detected for one or more of the larger spacings.

Using the arrangement of FIG. 3a as an illustration, it will be seen that any number of spatial offsets between the entry and collection regions may be used, for example from one up to ten or more offsets, with Raman features typically being detected during separate exposure time intervals for each offset, although simultaneous detection at multiple offsets may also or instead be used, for example to speed data collection while requiring more spectrometer resources. Although in FIG. 3a the entry region remains fixed relative to the sample and the collection region is moved, the entry region could be moved instead or as well as the collection region. The regions in FIG. 3a are essentially circular or elliptical in shape, typically determined by convenience of implementation of the delivery and collection optics, various other shapes may be used. In FIG. 3a none of the collection regions 16b-16d overlaps with the entry region, but some overlap maybe permitted.

In FIG. 3b, a concentric arrangement is used in which a central collection region 16 lies within a surrounding entry region 14a, which could be in the form of a continuous or broken annulus. This has an advantage in that the entry region is relatively large, and therefore can be provided using a lower intensity of illumination to avoid damaging the sample. Multiple spatial offsets can then be provided by varying the radius of the entry region, as depicted by concentric entry regions 14a and 14b. FIG. 3b shows an “inverse SORS” arrangement in which the, or each, entry region is generally disposed around, or form full or broken annuli around the central collection region. However, in other arrangements the central region may be the entry region, and the or each collection region is generally disposed around the central entry region.

FIG. 3c shows how an entry region 14 may be generally disposed around a central collection region 16 (or indeed vice versa), in which the entry region takes the form of a broken annulus, that is region of multiple discrete entry sub-regions each disposed at approximately the same distance or radius from the central collection region. Multiple such broken rings or broken annuli may be disposed at different radii from the central collection region to provide a plurality of spatial offsets as described above, and again the roles of the illustrated entry and collection regions may be reversed.

The in vivo tissues which provide the overlying tissue and sub-surface tissue described herein are typically diffusely scattering or turbid or strongly diffusing. The degree of such scattering will depend on the tissue, and may be defined in terms of transport length which is a length over which the direction of propagation of photon of probe light is randomized. The skilled person knows that transport length l* of diffusive scattering may be taken as being related to the mean free path by the expression:

$${l}^{⋆} = \frac{l}{1 - g}$$

where g is the asymmetry coefficient (average of the scattering angle over a large number of scattering events), and I is the mean free path. The diffuse scattering transport length for tissues of human or animal subjects which may be probed using the present invention may typically be of the order of 1 mm.

The one or more spatial offsets between the entry and collection regions used in embodiments of the invention may typically lie in a range of about 1 mm to about 50 mm, and more typically from about 3 mm to about 20 mm, and may be suitable for determining water content and other chemical characteristics in sub-surface tissues at depths beneath the surface of the overlying tissue in the range from about 1 mm to about 50 mm and more typically from about 3 mm to about 20 mm. Embodiments of the invention may be arranged to determine water content and other chemical characteristics at just one depth or depth profile, for example using a single spatial offset between the entry and collection regions, or may be arranged to determine such measures at each of multiple depths or depth profiles. Embodiments may also use a zero or null offset in order to determine such measures at the surface of the overlying tissue.

FIGS. 1, 3a and 3b depict entry and collection regions which are adjacent, proximal, or spaced apart on a surface of the overlying tissue which is largely planar or only moderately curved. Such an arrangement may be described as a backscatter configuration, because after penetrating into the tissue and undergoing Raman scattering in the sub-surface tissue, a photon of probe light is backscattered to the surface for collection by the collection optics. However, the entry and collection regions may also lie on parts of the surface which are far from coplanar, with substantially different surface normals, for example with normals in the region of 90 degrees apart, or even in the region of 180 degrees apart, or any other angle or range of angles.

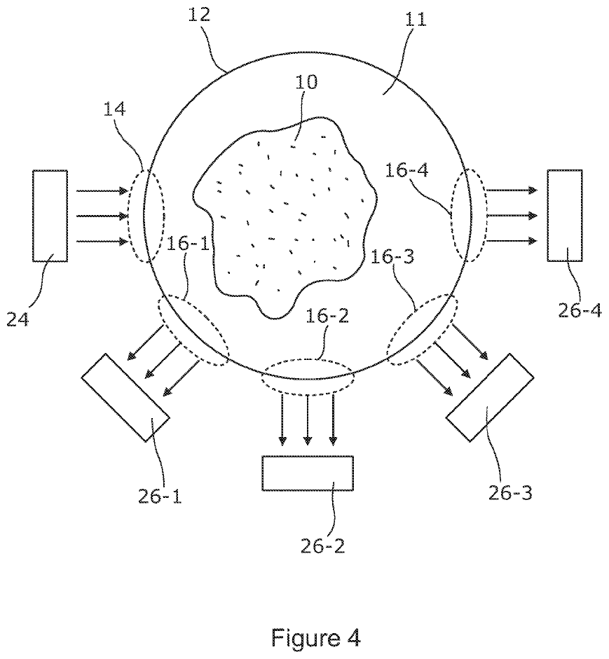

For example, the entry and collection regions may be disposed on opposite sides of a portion of the human or animal subject, or such that a sub-surface tissue 10 the properties of which are being determined lies directly between the entry and collection regions, and such arrangements may be described as transmission configurations. An example of a transmission configuration is provided in FIG. 4. In this figure, delivery optics 14 define an entry region 14 on one side of a portion of the subject. Four different positions for collection optics 26-1-26-4 are then shown in the figure to form four different collection regions 16-1-16-4. The collection region 16-4 is on an opposite side of the portion from the entry region, whereas the other collection regions 16-1-16-3 are spaced at angles of about 45, 90 and 135 degrees about the portion from the entry region. In such an arrangement, just one entry and one collection region could be used, or either or both of multiple entry and multiple collection regions may be used.

Transmission arrangements in which the sub-surface tissue 10 lies directly or approximately between the entry and collection regions may be of particular interest where the portion is small or thin, for example being only of the order of about 5 mm to about 50 mm in diameter or thickness for typical tissues, although larger diameters or spacings between entry and collection regions could be used if required.

Further discussion of transmission geometries and other details of such arrangements which can be used in embodiments of the present invention, to determine water content and other properties of a sub-surface tissue, can be found in the prior art including WO2007/113566, the contents of which is incorporated herein by reference in its entirety, to demonstrate how to arrange suitable transmission geometries for use in the present invention, and for all other purposes.

Note that, although the described techniques are particularly useful for detecting or measuring water content and other properties of a region of sub-surface tissue, it should be understood that some Raman signature of the overlying tissue and other nearby tissues optically coupled with the sub-surface tissue will typically also be mixed in with the signal from the sub-surface tissue, and cannot generally be excluded altogether in calculating desired parameters such as water content W. However, use of both the SORS and transmission Raman techniques described above allow for a significant degree of depth selection to take place, thereby reducing the contribution from the overlying and other incidental tissues. Furthermore, in many situations the variations in water content of the tissue being studied arise largely or strongly from variations of the sub-surface tissues of concern, as in the case of probing for sub-surface cancerous tissue in breasts and other body regions. In such situations detected variations in water content may largely be attributed to variations in the sub-surface tissue.

A wide range of tissues of human and animal subjects may be tested using embodiments of the present invention. Using a backscatter or SORS geometry, embodiments may be used to probe beneath skin for cancerous tissue and for other purposes across any part of the subject's body including limbs, trunk, neck, head, breasts and so forth. Transmission type geometries may be used for probing body parts where the spacing between opposing entry and collection regions can be reasonably small, for example for breasts especially when held in a clamped configuration, as well as in fingers, hands, feet, and other body areas where sufficient skin can be held between opposing entry and collection regions which may include for example skin on the stomach, legs, face, neck, prostate, and so forth.

Although the invention is particularly useful for in vivo and non-invasive use, experiments were carried out to demonstrate efficacy of the invention using ex vivo samples as described below, and in particular for these particular experiments using a transmission Raman geometry in which the collection region is disposed on an opposite side of the sample to the entry region.

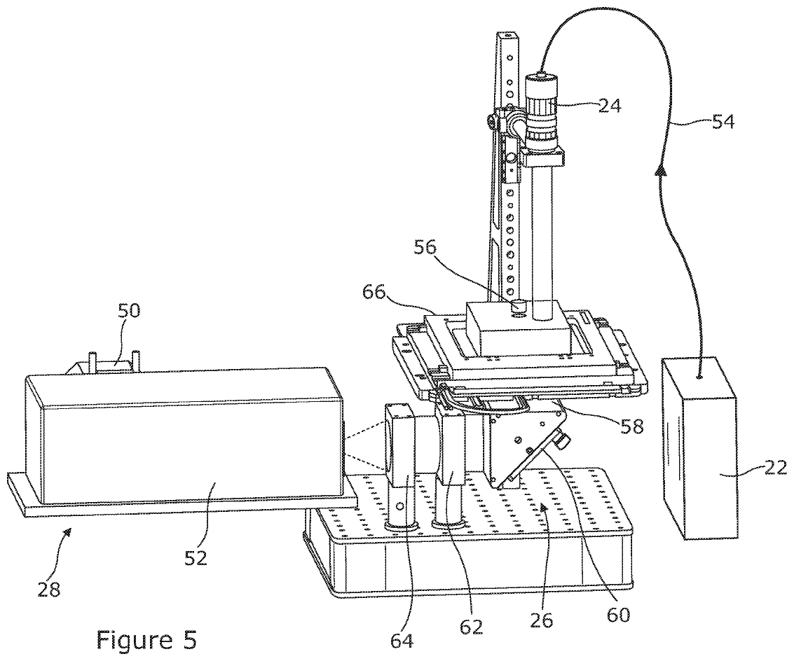

FIG. 5 illustrates in perspective view an optical arrangement used in these experiments, corresponding to the more generalised arrangement depicted in FIG. 1, and reference numerals are used in common with FIG. 1 where appropriate. In FIG. 5, the light source 22 is a 680 nm laser (IPS, New Jersey, USA), selected to enable high efficiency detection of the Raman bands of water spectra found in the high wavenumber region 33 by using a silicon-based CCD detector 50 forming part of spectrometer 28 which also includes a Holospec 1.8i spectrograph 52 (Kaiser optical). The 680 nm laser wavelength is rather low compared with conventional Raman sources which more typically operate in the near infrared, due to the requirement to detect features of the high wavenumber region and noting that CCDs generally have limited sensitivity beyond a wavelength of about 1000 nm.

A beam of probe light was delivered via a multimode fibre 54 to delivery optics 24 comprising a FC/PC-terminated zoom fibre collimator to form a beam of 5 mm diameter, spectrally filtered (680 nm laser band pass filter, Thorlabs) and directed onto an entry region on the surface of a sample 56, with an average optical beam power at the sample surface of about 160 mW. Collection optics 26 used to collect probe light from a collection region following scattering within the sample comprised 50 mm diameter optical elements including a 38.8 mm focal length lens 58, a mirror 60 to orient the collected light horizontally, and spectral edge long-pass filters 62 (Semrock Razor Edge, 830 nm) eliminating laser line followed by a lens 64 of 75 mm focal length imaging the collection region onto a slit of the spectrograph 52 and matching the spectrograph's numerical aperture.

The sample 56 was placed in a holder frame mounted on an automated x-y positioning stage 66 (provided by Standa, of Vilnius, Lithuania) to permit the sample to be scanned so that Raman measurements could be taken at a plurality of positions across the sample to form a map of related results, the plane of the map corresponding to the plane of the sample surface scanned by the delivery optics.

In substitution of the in vivo tissue discussed above, the sample 56 used in the present experiments was formed using fresh pork meat with a high content of muscle tissue, and chosen to avoid any potential content adulteration by additional chemical processing e.g. added water and preservatives. In a first set of measurements kinetics of natural drying at room temperature were monitored using as the sample a tissue block (5 mm thick, pork gammon) over a time span of 6-12 hours. Tissue weight loss, ascribed to reduction of water content, was monitored by electronic scales with stamped data points digitally recorded in parallel with Raman spectra. The aim was to establish that the Raman system is capable of detecting and quantifying tissue water content with adequate sensitivity. In these measurements the sample was placed on a 3D printed holder frame to ensure stability of tissue during time-lapse with enough clearance on both sides of the tissue sample for the incident laser beam and collection of Raman photons.

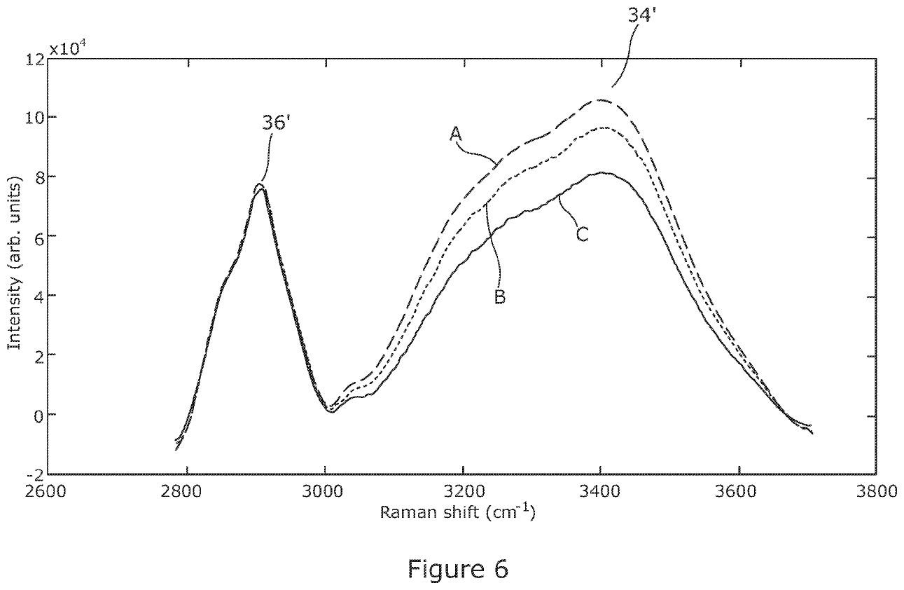

FIG. 6 depicts representative snapshots in the evolution of the high wavenumber Raman spectra of the tissue sample 56 while drying at room temperature. The plotted spectra are normalized such that the intensity of the Raman CH stretching bands peak 36′ remains constant. Three spectra are presented: spectrum A captured at the initial tissue weight followed by spectra B and C at the points of 17.5% and 32% reduction in tissue sample weight (assumed to be entirely due to water loss). The water percentage calculations are based on the net weight loss attributed to water evaporation in tissue and correlated with time stamp of the Raman spectra. A peak area calculation on whole of the Raman OH stretching bands peak 34′ from 3000 cm⁻¹ to 3700 cm⁻¹ indicates a change of only 5% in peak area associated with 17.5% real mass loss and a change of 8% in peak area related to a 32% real mass loss. This discrepancy is attributed to the fact that the evaporation occurs predominantly from surface areas of sample while transmission Raman spectroscopy is least sensitive to this zone, due to its inherent (typically several fold) bias towards central/inner parts of a sample (with lower drying rate) as opposed to very near-surface areas in this sampling geometry (with higher drying rate).

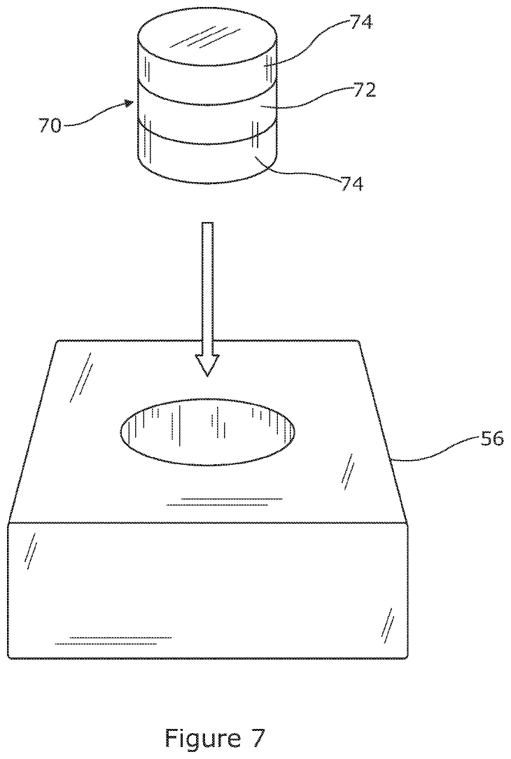

In the next set of measurements, the sample 56 included an inner volume of tissue with enhanced water content. As shown in FIG. 7, a fresh pork meat sample was cut to a thickness of 10 to 12 mm. A cylindrical core 70 about 20 mm in diameter was then cut from a central region of the sample 56. Once extracted, the core 70 was sliced into 3 smaller cylinder elements, and the middle cylinder element 72 with a thickness of around 5 mm was injected with water amount equivalent to an extra 20% of its weight and wrapped in a cling film to prevent water leakage into surrounding sample tissues. This water content increase amounts to a change in the water concentration in tissue from approximately 70% to 75% in the spiked central core volume. The core was then reassembled and inserted back into the sample to its original location. In this way, the middle core element 72 with enhanced water content corresponds to the sub-surface tissue 10 in FIG. 1, and one or both of the outer cylinder elements 74, along with surrounding uncured tissue of sample 46, corresponds to the overlying tissue 11.

The maps of measured water content W depicted in FIGS. 8a and 8b were formed by raster scanning the sample 46 past the delivery and collection optics, using the x-y stage, with a 3 mm step size over an area of about 20 cm² of the sample and acquisition times per map point of 1 second with 5 accumulations (i.e. 5 seconds total per point). The greyscales of the maps are in arbitrary units representing the water content as a ratio of the Raman OH and Raman CH signals. Data pre-processing consisting of cosmic ray signal removal, independent baseline subtraction and spectral peak analysis were performed using Matlab (®). Data interpretation and image construction was based on computing the ratio of the intensities of the spectral signal at the Raman OH stretching peak 34′ to that of the Raman CH stretching peak 36′, depicted as W=h₁/h₂ in FIG. 2.

The maps of water content were acquired both with the sample before water injection into the core 70 (FIG. 8a) and following water injection (FIG. 8b). The third map of FIG. 8c is a difference plot between the first two.

The Raman map (FIG. 8a) before water spiking illustrates the initial heterogeneity of the tissue water distribution across the mapped area born out, for example, by the presence of lipid rich areas in parts of sample. After this scan, the middle 5 mm disk of the removed core was injected with water, as illustrated in FIG. 7, in an amount equivalent to a 20% weight increase (raising effectively the water concentration in tissue from 70% to 75%). This loosely represents the presence of a cancer lesion which might contain even larger increases in the amount of water, as much as 20% increase in total water, i.e. from 50% to 70% water by mass. A second scan (FIG. 8b) was then performed using the same parameters as previously.

The presence of the added water is clearly revealed by subtracting the map of FIG. 8a from that of FIG. 8b, as shown in FIG. 8c. An analysis of the average pixel value from a central zone of nine pixels encompassing the maximum signal indicates average water elevation by 18% relative change in this core cylinder volume as a whole. This value is obtained as a percentage of the ratio between values in depicted in FIGS. 8c and 8a. As the spiked middle core represents only ˜40% of the entire cylinder volume this value translates to a 45% (0.18/0.4=0.45) increase in water mass alone in the middle spiked volume (not taking into account Raman signal bias to any specific depth). This equates to a calculated water signal in the spiked core of 77%, when taking into account the increased total mass of the central volume of the spiking water (as well as the original tissue mass). This matches remarkably well with the known spiked volume water concentration of 75% in tissue.

The results shown in FIGS. 8a to 8c therefore demonstrate the ability of the described techniques to measure water content of a sub-surface tissue through diffusely scattering overlying tissue. When evaluating the ability to detect a cancerous tissue or other tissue with elevated water content at depth, by using an artificially elevated water concentration (by 5% of total water concentration in the sub-surface tissue) the technique was able to distinguish and image clearly the water rich region within the sample. The describe techniques can therefore be used for potentially rapid localisation of cancer lesions which contain a higher water content, non-invasively and in vivo.

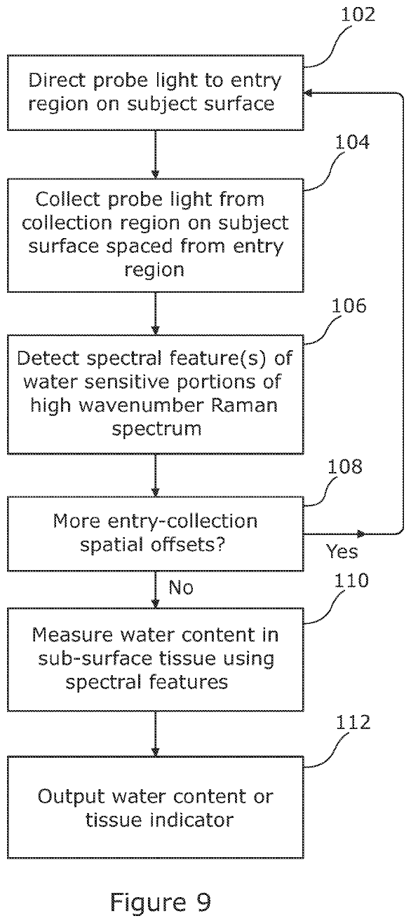

FIG. 9 provides a flow chart illustrating some methods for measuring water content in a sub-surface tissue as variously described above. In particular, the illustrated method may be used to measure, in vivo and optionally non-invasively, water content in a sub-surface tissue of a human or animal subject, through diffusely scattering overlying tissue.

At step 102, probe light is directed to an entry region on a surface of the overlying tissue, for example onto the skin of the subject, and at the same time, probe light is collected from a collection region on the surface of the overlying tissue, following scattering within the overlying tissue and within the sub-surface tissue. In particular, the collection region is spatially offset from the entry region, so that the collected light has been scattered within the sub-surface tissue to a significant extent. For example, at least 30% or at least 60%, or at least 90% of the collected light may have scattered within the sub-surface tissue, depending on the spatial offset and geometry between the entry and collection regions.

Notably therefore, the collected probe light comprises probe light inelastically scattered within the sub-surface tissue, and in particular comprises probe light scattered into the Raman OH stretching bands by water present in the sub-surface tissue, or other portions of the high wavenumber Raman region indicative of water content.

At step 106, first spectral features of the Raman OH stretching bands, or of other water sensitive portions of the high wavenumber Raman spectrum, are detected in the collected light. Other spectral features may also be detected, for example second spectral features of Raman CH stretching bands or other portions of the high wavenumber Raman spectrum suitable for use as reference portions of the spectrum, and one or more third spectral features of the lower wavenumber fingerprint region which can be used for detecting particular target chemical species other than water.

If required, the steps of directing 102 probe light to an entry region, collecting 104 probe light from a collection region, and detecting 106 spectral features of water sensitive portions of the high wavenumber Raman spectrum may be repeated for multiple different spacings between the entry and collection regions, as illustrated by decision step 108 which asks whether further spatial offsets should be used for the current measurement position. In this way, spectral features from different offsets can be combined to determine separate characteristics of the sub-surface tissue for one or more depths or distributions of depth beneath the surface, for example to select for a particular sub-surface tissue depth profile.

At step 110, one or more parameters of the sub-surface tissue can be determined from the spectral features. For example, water content can be measured from first spectral features, and optionally normalised or calibrated using the second spectral features which are insensitive to water content in the sub-surface tissue using the one or more first spectral features.

Finally, as step 112, one or more indicators of the sub-surface tissue such as water content, likelihood of being cancerous tissue, and so forth may be output as various described elsewhere in this document. An indicator of cancerous tissue may for example be calculated from a combination of a measure of water content using the first and second spectral features, and a measure of one or more other chemical components in the tissue using the third spectral features.

Although particular embodiments and applications of the invention have been described, it will be apparent to the skilled person that various modifications and alterations can be made without departing from the scope of the invention. For example, although the invention may be used to probe or measure water content non-invasively in subcutaneous tissues through overlying skin, it could instead be used to probe or measure water content in deeper skin layers such as the dermis layer through one or more overlying layers such as the epidermis.

1. A method of measuring, in vivo, water content in a sub-surface tissue of a human or animal subject, through diffusely scattering overlying tissue, comprising:
directing probe light to an entry region on a surface of the overlying tissue;
collecting said probe light from a collection region on the surface of the overlying tissue, the collection region being spatially offset from the entry region, the collected probe light comprising probe light inelastically scattered into the Raman OH stretching bands by water present in the sub-surface tissue;
detecting, in the collected probe light, one or more first spectral features of the probe light inelastically scattered into the Raman OH stretching bands; and
measuring water content in the sub-surface tissue using the one or more first spectral features.

2. The method of claim 1 wherein the Raman OH stretching bands extend at least from a wavenumber shift of about 2800 cm⁻¹ to a wavenumber shift of about 3700 cm⁻¹.

3. The method of claim 1 wherein the Raman OH stretching bands comprise a first spectral peak at about 3400 cm⁻¹, and the one or more first spectral features comprise one or more of: an area under at least a portion of the first spectral peak; and a magnitude of at least a portion of the first spectral peak.

4. The method of claim 1 wherein:
the collected probe light further comprises probe light inelastically scattered into the Raman CH stretching bands by C—H bonds present in the sub-surface tissue;
the method comprises detecting, in the collected probe light, one or more second spectral features of probe light inelastically scattered into the Raman spectral CH stretching bands; and
water content of the sub-surface tissue is measured using the one or more first spectral features and the one or more second spectral features.

5. The method of claim 4 wherein water content of the sub-surface tissue is measured using the one or more first spectral features normalised using the one or more second spectral features.

6. The method of claim 4 wherein the Raman CH stretching bands extend at least from a wavenumber shift of about 2800 cm⁻¹ to a wavenumber shift of about 3100 cm⁻¹.

7. The method of claim 4 wherein the Raman CH stretching bands provide a second spectral peak at about 2900 cm⁻¹, and the one or more second spectral features comprise one or more of: an area under at least a portion of the second spectral peak; and a magnitude of at least a portion of the second spectral peak.

8. The method of claim 1 wherein:
the probe light further comprises probe light inelastically scattered into the Raman fingerprint region by one or more chemical components of the sub-surface tissue;
the method comprises detecting, in the collected probe light, one or more third spectral features of probe light inelastically scattered into the Raman fingerprint region; and
measuring the chemical components of the sub-surface tissue using the one or more third spectral features.

9. The method of claim 8 wherein the Raman fingerprint region extends up to a wavenumber shift of about 1800 cm⁻¹.

10. The method of claim 1 wherein the entry and collection regions are disposed on opposite sides of the sub-surface tissue.

11. The method of claim 1 comprising separately detecting said one or more spectral features in the collected probe light for each of a plurality of different spatial offsets between said entry and collection regions.

12. The method of claim 11 wherein measuring water content of the sub-surface tissue from the spectral features comprises associating the spectral features from each of said plurality of different spatial offsets with a different depth or distribution of depth beneath the surface.

13. The method of claim 12 further comprising combining said spectral features from said different spatial offsets to determine a separate measure of water content for each of one or more depths or distributions of depth beneath the surface.

14. The method of claim 1 wherein the entry region comprises one or more segments which are located around a centrally disposed collection region.

15. The method of claim 14 wherein the entry regions comprise an annulus disposed around the collection region.

16. The method of claim 1 wherein the entry and collection regions are spatially offset by an offset in the range from 1 mm to 50 mm, and more preferably in the range from 3 mm to 20 mm.

17. The method of claim 1 wherein the sub-surface tissue is beneath the surface of the subject by least twice the diffuse scattering transport length of probe light in the sub-surface tissue.

18. The method of claim 1 wherein the sub-surface tissue is at least 2 mm beneath the surface of the subject.

19. The method of claim 1 further comprising determining an indication of the sub-surface tissue being cancerous from the measured water content.

20. The method of claim 19 wherein the determining an indication of the sub-surface tissue being cancerous also uses one or more spectral features detected in the collected probe light of the Raman fingerprint region.

21. The method of claim 20 wherein the one or more spectral features detected in the collected probe light of the Raman fingerprint region are indicative of one or more of: characteristic changes within nucleic and/or amino acids associated with dysfunctional tissues; lesion related calcifications; protein to lipid ratios associated with dysfunctional tissues; and protein conformations associated with dysfunctional tissues.

22. The method of claim 1 wherein measuring water content of the sub-surface tissue comprises determining an elevated water content of the sub-surface tissue.

23. The method of claim 1 further comprising generating a map of one or more of: measured water content in the sub-surface tissue; and an indication of the sub-surface tissue being cancerous, wherein the map corresponds to the plane of the surface.

24. The method of claim 23 wherein the map is generated from repeated measurements of water content or repeated indications of the sub-surface tissue being cancerous, taken at different positions across the surface.

25. Apparatus for measuring, in vivo, water content in a sub-surface tissue of a human or animal subject, through diffusely scattering overlying tissue, comprising:
a light source for generating probe light;
delivery optics arranged to direct probe light to an entry region on a surface of the overlying tissue;
collection optics arranged to collecting said probe light from a collection region on the surface of the overlying tissue, the collection region being spatially offset from the entry region;
a spectrometer arranged to detect, in the collected probe light, one or more first spectral features of a portion of the probe light inelastically scattered into Raman OH stretching bands by water present in the sub-surface tissue; and
an analyser arranged to determine water content in the sub-surface tissue from the one or more first spectral features.

26. The apparatus of claim 25 wherein:
the spectrometer is further arranged to detect, in the collected probe light, one or more second spectral features of a portion of the probe light inelastically scattered into Raman CH stretching bands by C—H bonds present in the sub-surface tissue; and
the analyser is arranged to determine water content in the sub-surface tissue from the one or more first spectral features and the one or more second spectral features.

27. The apparatus of claim 25 wherein:
the spectrometer is further arranged to detect, in the collected probe light, one or more third spectral features of a portion of the probe light inelastically scattered into the Raman fingerprint region by chemical components present in the sub-surface tissue; and
the analyser is arranged to detect the chemical components in the sub-surface tissue from the one or more third spectral features.

28. The apparatus of claim 25 wherein the delivery optics and the collection optics are arranged such that, in use, they lie on opposite sides of the sub-surface tissue.

29. The apparatus of claim 25 further comprising an offset driver arranged to provide a plurality of different offset spacings between the entry and collection regions.

30. The apparatus of claim 29 arranged to separately detect said one or more spectral features in the collected probe light for each of a plurality of different spatial offsets between said entry and collection regions, and wherein the analyser is arranged to measure water content in the sub-surface tissue from the spectral features by associating the spectral features from each of said plurality of different spatial offsets with a different depth or distribution of depth beneath the surface.

31. The apparatus of claim 25 wherein the analyser is arranged to determine whether the sub-surface tissue has an elevated water content.

32. The method of claim 1 wherein the probe light directed to the entry region is laser light with a wavelength of between 630 nm and 720 nm.

33. The method of claim 1 wherein the sub-surface tissue is a subcutaneous tissue of the human or animal subject, and the diffusely scattering overlying tissue comprises skin of the subject.

34. The apparatus of claim 25 wherein the probe light directed to the entry region is laser light with a wavelength of between 630 nm and 720 nm.

35. The apparatus of claim 25 wherein the sub-surface tissue is a subcutaneous tissue of the human or animal subject, and the diffusely scattering overlying tissue comprises skin of the subject.
